(19) United States
(12) Patent Application Publication
(10) Pub. No.: US 2017/0181762 A1
(43) Pub. Date: Jun. 29, 2017

(54) Liquid Ejection Control Apparatus, Liquid Ejection System, And Control Method

(71) Applicant: Seiko Epson Corporation, Tokyo (JP)

(72) Inventor: Junichi Karasawa, Shimosuwa-machi (JP)

(73) Assignee: Seiko Epson Corporation, Tokyo (JP)

(21) Appl. No.: 15/325,485

(22) PCT Filed: Jul. 2, 2015
(86) PCT No.: PCT/JP2015/003343

§ 371 Date: Jan. 11, 2017

(30) Foreign Application Priority Data
Jul. 11, 2014 (JP) 2014-143097

(51) Int. Cl. A61B 17/3203; B26F 3/00

(52) U.S. Cl. CPC A61B 17/3203; B26F 3/004

(57) ABSTRACT

In a liquid ejection control apparatus, an operation unit includes adjustment levers for changing a rising frequency, voltage amplitude, and the like of a drive voltage waveform applied to a piezoelectric element. In a control unit, a rising frequency setting section sets a rising frequency in response to an input operation on a rising frequency adjustment lever so as to make constant a change amount regarding momentum related to a pulsed liquid jet ejected from a liquid ejection device and the change amount per unit operation level for the rising frequency adjustment lever in a state in which voltage amplitude of the drive voltage waveform is set to a predetermined value.

### TECHNICAL FIELD

The present invention relates to a liquid ejection control apparatus and the like controlling a liquid ejection device which ejects a liquid in a pulse form by using a piezoelectric element.

### BACKGROUND ART

There is a technique of cutting a cutting target object by ejecting a liquid in a pulse form. The liquid ejected in a pulse form is a liquid jet flow which is periodically or non-periodically ejected from a nozzle in a pulsating manner, and is referred to as a “pulsed liquid jet” as appropriate in the present specification.

A pulsed liquid jet may be variously applied, and, for example, PTL 1 has proposed a technique in which the pulsed liquid jet is used for surgery in a medical field. In this case, a cutting target object is a living tissue, and a liquid is physiological saline.

### CITATION LIST

### Patent Literature

PTL 1: JP-A-2005-152127

### SUMMARY OF INVENTION

### Technical Problem

As one of mechanisms generating a pulsed liquid jet, there is a mechanism using a piezoelectric element. The mechanism applies a pulsed drive voltage to a piezoelectric element so that the piezoelectric element generates instantaneous pressure, and thus ejects the liquid in a pulse form. Thus, the strength of the pulsed liquid jet is changed by controlling a drive voltage applied to the piezoelectric element.

Therefore, there may be a technique in which a characteristic value of a drive voltage applied to a piezoelectric element, for example, the amplitude (which is voltage amplitude and can be said to the magnitude of the drive voltage) of a drive voltage waveform is indicated by using an operation unit such as an operation dial or an operation button, and thus the strength of a pulsed liquid jet is changed.

However, it has been found that, even if the characteristic value of the drive voltage indicated by the operation unit is changed, there is a case where a cutting aspect such as a cut depth or a cut volume of a cutting target object may not be changed as intended by a user. As will be described later in detail, it has been found that, for example, even if the user changes the voltage amplitude to twice or four times, or a half or a quarter, a cut depth or a cut volume is not necessarily changed in proportion thereto. In a case where a pulsed liquid jet is used for surgery, there may be a problem in that working corresponding to an operator's operation sense is not performed.

The invention has been made in light of the above-described problems, and an object thereof is to provide a technique capable of setting the strength of a pulsed liquid jet as intended by a user so as to improve convenience.

### Solution to Problem

In order to solve the above-described problem, a first invention is directed to a liquid ejection control apparatus controlling a liquid ejection device which ejects a liquid in a pulse form by using a piezoelectric element, the liquid ejection control apparatus including an operation unit that is used to perform a changing operation on one of the amplitude of a drive voltage waveform applied to the piezoelectric element, and an index value related to rising of the drive voltage waveform; and a control unit that performs control of setting a value of the one in response to an input operation on the operation unit so as to make constant a change amount regarding momentum or kinetic energy related to a pulsed liquid jet ejected from the liquid ejection device and the change amount per unit operation level for the operation unit in a state in which the other of the amplitude of the drive voltage waveform and the index value related to rising of the drive voltage waveform is set to a predetermined value.

Another invention may be configured as a control method for a liquid ejection device which ejects a liquid in a pulse form by using a piezoelectric element, the control method including detecting an input operation on an operation unit that is used to perform a changing operation on one of the amplitude of a drive voltage waveform applied to the piezoelectric element, and an index value related to rising of the drive voltage waveform; and setting a value of the one in response to an input operation on the operation unit so as to make constant a change amount regarding momentum or kinetic energy related to a pulsed liquid jet ejected from the liquid ejection device and the change amount per unit operation level for the operation unit in a state in which the other of the amplitude of the drive voltage waveform and the index value related to rising of the drive voltage waveform is set to a predetermined value.

According to the first invention and the like, there is the operation unit that is used to perform a changing operation on one of the amplitude of a drive voltage waveform applied to the piezoelectric element, and an index value related to rising of the drive voltage waveform. A value of the one is set in response to an input operation on the operation unit so as to make constant a change amount regarding momentum or kinetic energy related to a pulsed liquid jet and the change amount per unit operation level for the operation unit in a state in which the other of the amplitude of the drive voltage waveform and the index value related to rising of the drive voltage waveform is set to a predetermined value.

As will be described later, a cut depth or a cut volume is highly correlated with momentum and kinetic energy related to a pulsed liquid jet. Since a change amount of the momentum or the kinetic energy is constant per unit operation level for the operation unit, it is possible to realize a cut depth or a cut volume to be suitable for a user's intention or operation sense and thus to improve convenience.

A second invention is directed to the liquid ejection control apparatus according to the first invention, which further includes a display control unit that performs control of displaying the present value of momentum and kinetic energy related to the pulsed liquid jet.

According to the second invention, it is possible to display the present value of momentum or kinetic energy related to a pulsed liquid jet. Thus, a user can visually recognize an index indicating the present strength of a desired pulsed liquid jet. Therefore, it is possible to further improve convenience.

A third invention is directed to the liquid ejection control apparatus according to the first or second invention, in which a correspondence relationship between the amplitude of the drive voltage waveform applied to the piezoelectric element and the index value related to rising of the drive voltage waveform, causing the change amount per unit operation level to be constant, is defined for each type of liquid ejection device, and the control unit performs control on the basis of the correspondence relationship corresponding to the type of liquid ejection device.

According to the third invention, even in a case where the type of liquid ejection device is changed, it is possible to perform appropriate control corresponding to the type of liquid ejection device which is a control target.

A fourth invention is directed to the liquid ejection control apparatus according to any one of the first to third inventions, in which the liquid ejection device is controlled so that momentum of the pulsed liquid jet is equal to or less than 0.1 millinewton seconds (mNs), or kinetic energy of the pulsed liquid jet is equal to or less than 100 millijoules (mJ).

According to the fourth invention, it is possible to control the liquid ejection device within a range in which the momentum of the pulsed liquid jet is equal to or less than 0.1 mNs, or the kinetic energy thereof is equal to or less than 100 mJ. Therefore, the liquid ejection control apparatus is suitable to cut soft materials, for example, a living tissue, food, a gel material, and a resin material such as rubber or plastic.

A fifth invention is directed to the liquid ejection control apparatus according to any one of the first to fourth inventions, in which the liquid ejection device is controlled so that a living tissue is cut with the pulsed liquid jet.

According to the fifth invention, it is possible to control the strength of a pulsed liquid jet suitable for a surgery application, for example.

A sixth invention is directed to the liquid ejection control apparatus according to any one of the first to fifth inventions, in which the index value related to the rising is represented by time or a frequency related to the rising of the drive voltage waveform.

According to the sixth invention, it is possible to cause an index value related to rising to be represented by time or a frequency related to rising of a voltage.

A seventh invention is directed to a liquid ejection system including the liquid ejection control apparatus according to any one of the first to sixth inventions; a liquid ejection device; and a liquid feeding pump device.

According to the seventh invention, it is possible to implement a liquid ejection system capable of achieving the operations and effects of the first to sixth inventions.

### BRIEF DESCRIPTION OF DRAWINGS

FIG. 1 is a diagram illustrating the entire configuration example of a liquid ejection system.

FIG. 2 is a diagram illustrating an internal structure of a liquid ejection device.

FIG. 3 are diagrams illustrating a drive voltage waveform for a piezoelectric element corresponding to one cycle and a liquid flow velocity waveform in a liquid ejection opening.

FIG. 4 are diagrams respectively illustrating mass flow flux Jm, momentum flow flux Jp, and energy flow flux Je.

FIG. 5 are diagrams illustrating flow velocity waveforms of a main jet used in simulation for a destruction state of a cutting target object.

FIG. 6 are diagrams illustrating simulation results (cut depths).

FIG. 7 are diagrams illustrating simulation results (cut volumes).

FIG. 8 are diagrams illustrating simulation results of flow velocity waveforms of a main jet.

FIG. 9 is a diagram illustrating a correspondence relationship among momentum P, a rising frequency, and the voltage amplitude.

FIG. 10 is a diagram illustrating a correspondence relationship among energy E, a rising frequency, and the voltage amplitude.

FIG. 11 is a block diagram illustrating a functional configuration example of a liquid ejection control apparatus in Example 1.

FIG. 12 is a diagram illustrating a data configuration example of a momentum correspondence table in Example 1.

FIG. 13 is a flowchart illustrating a flow of a process performed by a control unit when a pulsed liquid jet is ejected.

FIG. 14 is a diagram illustrating a display screen example of a display unit.

FIG. 15 is a block diagram illustrating a functional configuration example of a liquid ejection control apparatus in Example 2.

FIG. 16 is a diagram illustrating a data configuration example of a momentum correspondence table in Example 2.

FIG. 17 is a block diagram illustrating a functional configuration example of a liquid ejection control apparatus in Example 3.

FIG. 18 is a diagram illustrating a data configuration example of an energy correspondence table in Example 3.

FIG. 19 is a block diagram illustrating a functional configuration example of a liquid ejection control apparatus in Example 4.

FIG. 20 is a diagram illustrating a data configuration example of an energy correspondence table in Example 4.

### DESCRIPTION OF EMBODIMENTS

Hereinafter, a description will be made of embodiments of a liquid ejection control apparatus and a liquid ejection control method according to the invention. The invention is not limited to the embodiments described below, and embodiments to which the invention is applicable are not limited to the embodiments described below. The same portions are given the same reference numerals throughout the drawings.

### [Entire Configuration]

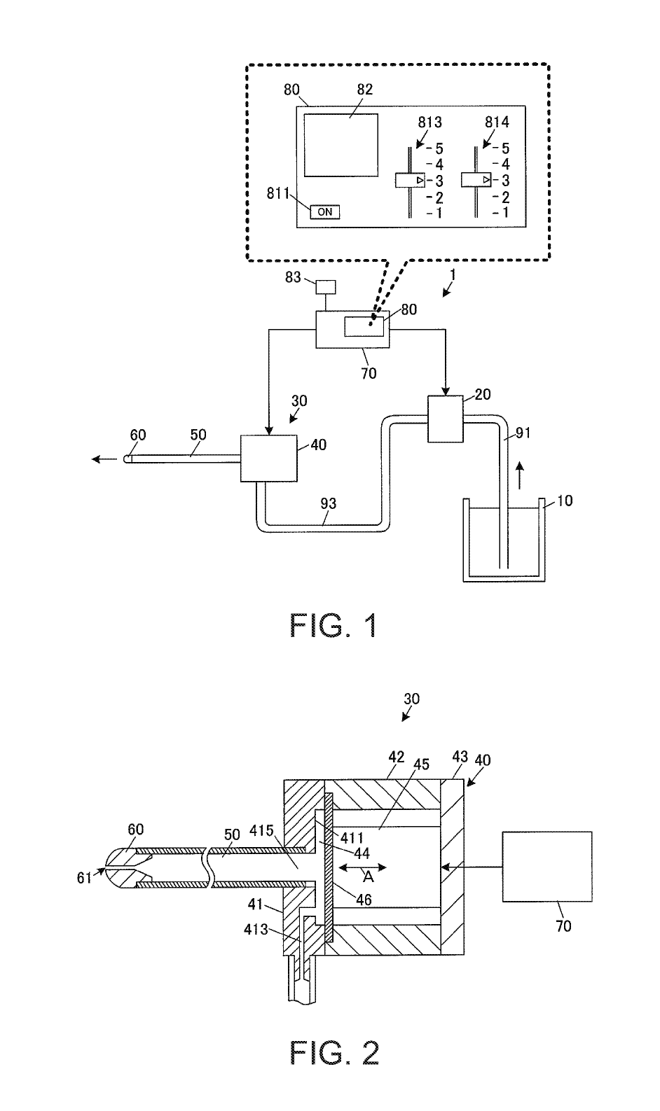

FIG. 1 is a diagram illustrating the entire configuration example of a liquid ejection system 1 in the present embodiment. The liquid ejection system 1 is used for applications such as surgery with a soft material, for example, a living tissue as a cutting target object, food processing with food as a cutting target object, processing of a gel material, and cutting processing of a resin material such as rubber or plastic, and ejects a pulsed liquid jet whose momentum is equal to or less than 0.1 millinewton seconds (mNS), or whose kinetic energy is equal to or less than 100 millijoules (mJ) so as to cut a cutting target object. Hereinafter, a case will be exemplified in which the liquid ejection system 1 is used for a surgery application and performs incision, excision, or crushing (these are collectively referred to as “cutting”) of the affected part (living tissue).

As illustrated in FIG. 1, the liquid ejection system 1 includes a container 10 accommodating a liquid, a liquid feeding pump 20, a liquid ejection device 30 which ejects the liquid toward a cutting target object (a living tissue in the present embodiment) in a pulse form, and a liquid ejection control apparatus 70.

In the liquid ejection system 1, the liquid ejection control apparatus 70 is provided with an operation panel 80 which is operated by an operator during surgery. The operation panel 80 is provided with a button switch 811 for switching between turning-on and turning-off of power supply; a lever switch 813 which allows lever positions in five steps, provided with scales such as “1” to “5”, to be selected; a repetition frequency setting lever switch 814 which allows lever positions in five steps, provided with scales such as “1” to “5”, to be selected; and a liquid crystal monitor 82. The liquid ejection control apparatus 70 is provided with a pedal switch 83 for switching between ejection starting and ejection stoppage of a pulsed liquid jet by the operator treading thereon.

The container 10 accommodates a liquid such as water, physiological saline, or a chemical liquid. The liquid feeding pump 20 supplies the liquid accommodated in the container 10 to a pulse flow generator 40 of the liquid ejection device 30 at predetermined pressure or a predetermined flow rate at all times via connection tubes 91 and 93.

The liquid ejection device 30, which is a portion (handpiece) operated by the operator holding in his or her hand during surgery, includes the pulse flow generator 40 which gives pulsation to the liquid supplied from the liquid feeding pump 20 so as to generate a pulse flow, and a pipe-shaped ejection tube 50, and ejects the pulse flow generated by the pulse flow generator 40 from a liquid ejection opening 61 provided at a nozzle 60 through the ejection tube 50 as a pulsed liquid jet.

Here, the pulse flow indicates a pulsative flow of the liquid which considerably and rapidly changes temporally or spatially in a periodic or non-periodic manner in terms of a flow velocity or pressure thereof. Similarly, ejecting a liquid in a pulse form indicates pulsative ejection of the liquid in which a flow velocity of the liquid passing through the nozzle considerably changes in a periodic or non-periodic manner. In the present embodiment, a case of ejecting a pulsed liquid jet generated by applying periodic pulsation to a steady flow is exemplified, but the invention is also applicable to intermittent ejection of a pulsed liquid jet in which ejection and non-ejection of a liquid are repeatedly performed in a periodic or non-periodic manner.

FIG. 2 is a diagram illustrating a cut surface obtained by cutting the liquid ejection device 30 along a liquid ejection direction. Vertical and horizontal scales of members or portions illustrated in FIG. 2 are different from actual ones for convenience of illustration. As illustrated in FIG. 2, the pulse flow generator 40 is configured of a piezoelectric element 45 and a diaphragm 46 which change a volume of a pressure chamber 44 and which are disposed in a tubular internal space formed by a first case 41, a second case 42, and a third case 43. The respective cases 41, 42 and 43 are joined together and are thus integrally formed at surfaces facing each other.

The diaphragm 46 is a disk-shaped metal thin plate, and an outer circumferential portion thereof is interposed and fixed between the first case 41 and the second case 42. The piezoelectric element 45 is, for example, a laminated piezoelectric element, and has one end fixed to the diaphragm 46 between the diaphragm 46 and the third case 43, and the other end fixed to the third case.

The pressure chamber 44 is a space surrounded by the diaphragm 46, and a depression 411 formed on a surface facing the diaphragm 46 of the first case 41. The first case 41 is provided with an inlet channel 413 and an outlet channel 415 which communicate with the pressure chamber 44. An inner diameter of the outlet channel 415 is larger than an inner diameter of the inlet channel 413. The inlet channel 413 is connected to the connection tube 93 and introduces a liquid supplied from the liquid feeding pump 20 into the pressure chamber 44. One end of the ejection tube 50 is connected to the outlet channel 415, and thus the liquid flowing in the pressure chamber 44 is introduced into the ejection tube 50. The nozzle 60 having a liquid ejection opening 61 which has an inner diameter smaller than an inner diameter of the ejection tube 50 is inserted into the other end (front end) of the ejection tube 50.

In the liquid ejection system 1 configured in the above-described way, the liquid accommodated in the container 10 is supplied to the pulse flow generator 40 via the connection tube 93 at predetermined pressure or a predetermined flow rate by the liquid feeding pump 20 under the control of the liquid ejection control apparatus 70. On the other hand, if a drive signal is applied to the piezoelectric element 45 under the control of the liquid ejection control apparatus 70, the piezoelectric element 45 is expanded or contracted (an arrow A in FIG. 2). The drive signal applied to the piezoelectric element 45 is repeatedly applied at a predetermined repetition frequency (for example, several tens of Hz to several hundreds of Hz), and thus expansion and contraction of the piezoelectric element 45 are repeatedly performed for each cycle. Consequently, pulsation is applied to the steady flow liquid flowing in the pressure chamber 44, and thus a pulsed liquid jet is repeatedly ejected from the liquid ejection opening 61.

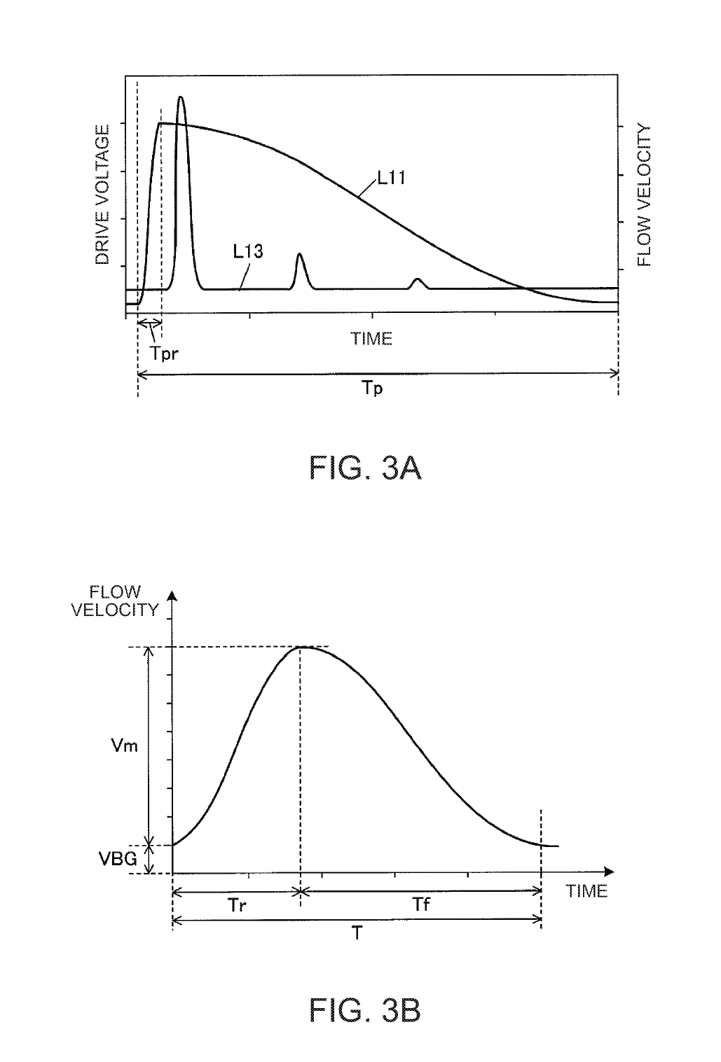

FIG. 3(a) is a diagram illustrating an example of a driving voltage waveform L11 of a drive signal corresponding to one cycle applied to the piezoelectric element 45, and also illustrates a flow velocity waveform L13 of a liquid in the liquid ejection opening 61. Tp indicates a repetition cycle (time corresponding to one cycle of a drive voltage waveform), and an inverse number thereof is the above-described repetition frequency.

FIG. 3(b) is a diagram obtained by extracting a main peak portion with the maximum flow velocity from among peaks of the flow velocity waveform L13 illustrated in FIG. 3(a). The repetition cycle Tp is about 1 millisecond (ms) to 100 ms, and time (rising time) Tpr for the drive voltage waveform to rise to the maximum voltage is 10 microseconds (μs) to 1000 μs.

The repetition cycle Tp is set to be longer than the rising time Tpr. In a case where an inverse number of twice the rising time is a rising frequency, the repetition frequency is set to be lower than twice the rising frequency.

For example, if the piezoelectric element 45 is expanded when a positive voltage is applied thereto, the piezoelectric element 45 is rapidly expanded at the rising time Tpr, and thus the diaphragm 46 is pushed by the piezoelectric element 45 so as to be bent toward the pressure chamber 44 side. If the diaphragm 46 is bent toward the pressure chamber 44 side, the volume of the pressure chamber 44 is reduced, and thus the liquid in the pressure chamber 44 is pushed out of the pressure chamber 44. Here, since the inner diameter of the outlet channel 415 is larger than the inner diameter of the inlet channel 413, fluid inertance and fluid resistance of the outlet channel 415 are less than fluid resistance of the inlet channel 413. Therefore, most of the liquid pushed out of the pressure chamber 44 due to rapid expansion of the piezoelectric element 45 is introduced into the ejection tube 50 through the outlet channel 415, and is ejected at a high speed as pulsed liquid droplets, that is, a pulsed liquid jet through the liquid ejection opening 61 having the diameter smaller than the inner diameter of the outlet channel.

The drive voltage increases to the maximum voltage, and then slowly decreases. At this time, the piezoelectric element 45 is contracted for a longer time than the rising time Tpr, and thus the diaphragm 46 is pulled to the piezoelectric element 45 so as to be bent toward the third case 43 side. If the diaphragm 46 is bent toward the third case 43 side and thus the volume of the pressure chamber 44 is increased, the liquid is introduced into the pressure chamber 44 from the inlet channel 413.

Since the liquid feeding pump 20 supplies the liquid to the pulse flow generator 40 at predetermined pressure or a predetermined flow rate, if the piezoelectric element 45 does not perform expansion and contraction operations, the liquid (steady flow) flowing in the pressure chamber 44 is introduced into the ejection tube 50 through the outlet channel 415, and is ejected from the liquid ejection opening 61. The ejected flow is a liquid flow at a constant and low speed, and may thus be regarded as a steady flow.

### [Principle]

Fundamental values indicating features of a pulsed liquid jet are the drive voltage waveform. L11 in FIG. 3(a) and the flow velocity waveform L13 of a jet corresponding to a single pulse in the liquid ejection opening 61. Above all, a flow velocity waveform having the highest peak (a jet in a head wave) generated right after rising of the drive voltage, is focused. FIG. 3(b) is an enlarged view of this waveform. Other low peaks are caused by jets which are incidentally ejected since a pressure changing wave occurring in the pressure chamber 44 during expansion of the piezoelectric element 45 reflects and reciprocates in the ejection tube 50, but a destruction state of a cutting target object, that is, a cut depth or a cut volume of the cutting target object is determined by a jet in a head wave (main jet) with the highest flow velocity.

In a case where a cut depth or a cut volume of the cutting target object is to be changed by changing the strength of a pulsed liquid jet, a drive voltage waveform for the piezoelectric element 45 is controlled. There may be a method of controlling the drive voltage waveform by the operator designating a rising frequency of the drive voltage waveform or amplitude (voltage amplitude) of the drive voltage waveform as a voltage characteristic value. The rising frequency mentioned here is one of index values associated with rising of a drive voltage, and is defined as an inverse number of a value which is twice the rising time Tpr. For example, there may be a method in which the operator designates a rising frequency in a state in which a voltage amplitude is fixed, and designates a voltage amplitude in a state in which a rising frequency is fixed. This is because the voltage amplitude or the rising frequency (rising time Tpr) greatly influences a flow velocity waveform of the main jet. A drive voltage which is slowly decreasing after increasing to the maximum voltage does not greatly influence the flow velocity waveform of the main jet. For example, if the rising frequency is heightened, or the voltage amplitude is increased, it is considered that a cut depth and a cut volume are increased in proportion thereto.

However, it has been proved that an actually obtained cut depth or cut volume of a cutting target object may not necessarily be changed in accordance with a change in the voltage characteristic value, and thus convenience may deteriorate. For example, there is a case where the operator increases the voltage amplitude to twice, but a cut depth or a cut volume may not be increased as expected, or decreases the voltage amplitude to a half, but a cut depth or a cut volume may not be reduced as expected. Thus, a situation may occur in which a cut depth or a cut volume desired by the operation is not obtained. This causes a problem of increasing surgery time.

Therefore, focusing on a flow velocity waveform of the main jet, correlations of a cut depth and a cut volume with several parameters determined by the flow velocity waveform of the main jet were examined. This is because, if a parameter highly correlated with a cut depth or a cut volume is found, the piezoelectric element 45 can be controlled with a drive voltage waveform which is optimal for achieving a cut depth or a cut volume corresponding to the operator's operation sense.

For this, first, on the basis of a flow velocity waveform v [m/s] of the main jet in the liquid ejection opening 61, mass flow flux [kg/s], momentum flow flux [N], and energy flow flux [W] of the main jet passing through the liquid ejection opening 61, were examined. The mass flow flux is mass [kg/s] per unit time of a liquid passing through the liquid ejection opening 61. The momentum flow flux is momentum [N] per unit time of a liquid passing through the liquid ejection opening 61. The energy flow flux is energy [W] per unit time of a liquid passing through the liquid ejection opening 61. The energy indicates kinetic energy, and will be hereinafter abbreviated to “energy”.

In the liquid ejection opening 61, a liquid is released to a free space, and thus pressure may be regarded as being “0”. A velocity of the liquid in a direction (a diameter of the liquid ejection opening 61) orthogonal to a jet ejection direction may also be regarded as being “0”. Assuming that there is no velocity distribution of a liquid in the diameter direction of the liquid ejection opening 61, mass flow flux Jm [kg/s], momentum flow flux Jp [N], and energy flow flux Je [W] of the liquid passing through the liquid ejection opening 61 may be respectively obtained according to the following Equations (1), (2) and (3). S [m²] indicates a nozzle sectional area, and ρ [kg/m³] indicates a working fluid density.

Jm=S·ρ·v (1)

Jp=S·ρ·v² (2)

Je=½·ρ·S·v³ (3)

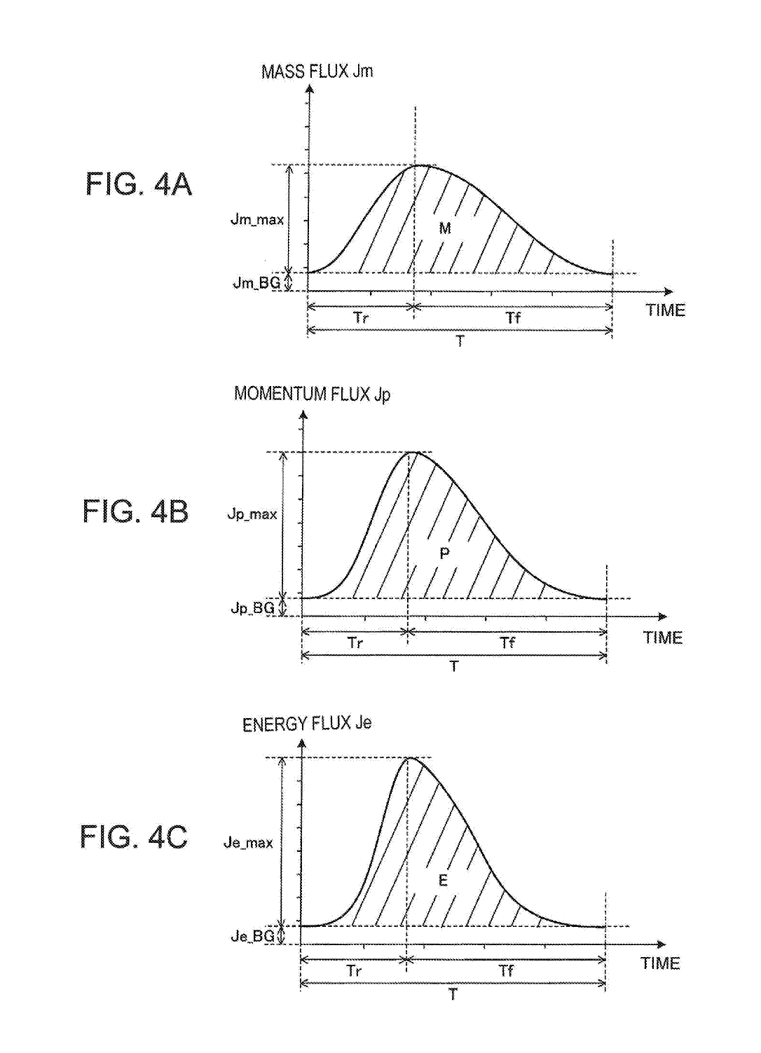

FIG. 4 are diagrams respectively illustrating mass flow flux Jm (FIG. 4(a)), momentum flow flux Jp (FIG. 4(b)), and energy flow flux Je (FIG. 4(c)) obtained on the basis of the flow velocity waveform of the main jet illustrated in FIG. 3(b). If each of the mass flow flux Jm, the momentum flow flux Jp, and the energy flow flux Je is integrated over time (duration) T from rising to falling of the flow velocity waveform of the main jet, mass, momentum, and energy of a liquid ejected from the liquid ejection opening 61 as the main jet can be obtained.

Each value of mass flow flux Jm, the momentum flow flux Jp, the energy flow flux Je, the mass, the momentum, and the energy calculated in the above-described way may determine a cut depth and a cut volume related to a jet corresponding to a single pulse. However, each of the above physical quantities includes a quantity corresponding to a steady flow, and it is noted that a value thereof is obtained by subtracting an attribution of the steady flow.

Therefore, regarding the mass flow flux Jm illustrated in FIG. 4(a), two parameters are defined, such as the maximum mass flow flux Jm_max [kg/s] obtained by subtracting mass flow flux Jm_BG [kg/s] of a steady flow from a peak value (maximum value) of the mass flow flux Jm, and outflow mass M [kg], hatched in FIG. 4(a), obtained by excluding an amount corresponding to the steady flow from mass of a liquid flowing out of the liquid ejection opening 61 as the main jet. The outflow mass M is expressed by the following Equation (4).

M=∫(Jm−Jm_BG)dt (4)

Regarding the momentum flow flux Jp illustrated in FIG. 4(b), two parameters are defined, such as the maximum momentum flow flux Jp_max [N] obtained by subtracting momentum flow flux Jp_BG [N] of a steady flow from a peak value (maximum value) of the momentum flow flux Jp, and momentum P [Ns], hatched in FIG. 4(b), obtained by excluding an amount corresponding to the steady flow from momentum of a liquid flowing out of the liquid ejection opening 61 as the main jet. The momentum P is expressed by the following Equation (5).

P=∫(Jp−Jp_BG)dt (5)

Regarding the energy flow flux Je illustrated in FIG. 4(c), two parameters are defined, such as the maximum energy flow flux Je_max [W] obtained by subtracting energy flow flux Je_BG [W] of a steady flow from a peak value (maximum value) of the energy flow flux Je, and energy E [J], hatched in FIG. 4(c), obtained by excluding an amount corresponding to the steady flow from energy of a liquid flowing out of the liquid ejection opening 61 as the main jet. The energy E is expressed by the following Equation (6).

E=∫(Je−Je_BG)dt (6)

Here, the integration section in each of the above Equations (4), (5) and (6) is time (duration) T from rising to falling of the main jet in the flow velocity waveform.

By using numerical value simulation, to what extent each of the six parameters such as the maximum mass flow flux Jm_max, the outflow mass M, the maximum momentum flow flux Jp_max, the momentum P, the maximum energy flow flux Je_max, and the energy E is correlated with a cut depth and a cut volume was examined.

Here, a pulsed liquid jet is a fluid, and a cutting target object is a soft elastic body. Therefore, in order to perform simulation for a destruction behavior of the cutting target object using the pulsed liquid jet, an appropriate destruction threshold value is set on the soft elastic body side, and then so-called interaction analysis (fluid structure interaction (FSI) analysis) of the fluid and a structure (here, the soft elastic body) is required to be performed. Computation methods in simulation may include a finite element method (FEM), a method using a particle method whose representative is a smoothed particle hydrodynamics (SPH), and a method of combining the finite element method with the particle method. An applied method is not particularly limited. Thus, although not described in detail, an optimal method was selected by taking into consideration stability of an analysis result, computation time, and the like, and the simulation was performed.

When the simulation was performed, a diameter of the liquid ejection opening 61=0.15 mm, and a standoff distance (a distance from the liquid ejection opening 61 to a surface of the cutting target object)=0.5 mm were set. Assuming that the cutting target object was a soft elastic body having a flat surface, a Mooney-Rivlin super-elastic body having an elastic modulus of about 9 kPa (about 3 kPa in terms of shear modulus) in terms of Young's modulus was used as a physical model thereof. Equivalent deviation strain=0.7 was used in the destruction threshold value.

Regarding flow velocity waveforms of the main jet, various flow velocity waveforms of the main jet were assumed, and a total of flow velocity waveforms of 27 types were prepared by changing amplitude (the maximum value of flow flux) of three types in a range of 12 m/s to 76 m/s and changing duration of three type in a range of 63 μs to 200 μs, with respect to each of waveforms of three types such as a sine wave, a triangular wave, and a rectangular wave. A flow velocity of a steady flow was 1 m/s.

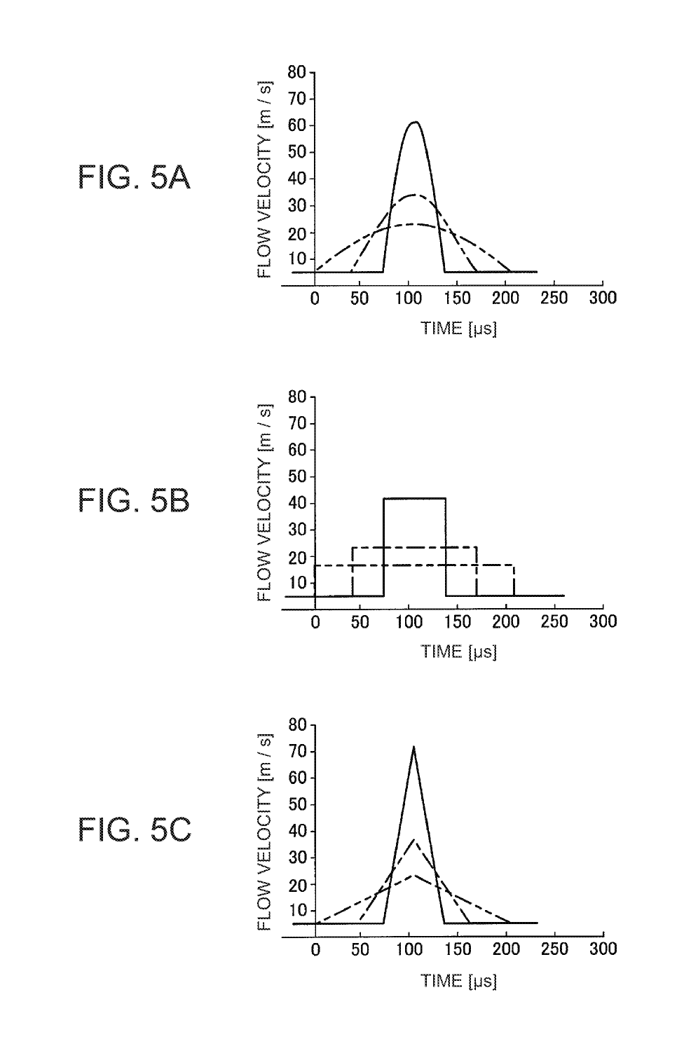

FIG. 5 respectively illustrate a sine wave (FIG. 5(a)), a rectangular wave (FIG. 5(b)), and a triangular wave (FIG. 5(c)) provided as flow velocity waveforms of the main jet in the simulation, in which a solid line indicates a case where the duration is 63 μs, a dot chain line indicates a case where the duration is 125 μs, and a two-dot chain line indicates a case where the duration is 200 μs. The prepared waveforms were provided as flow velocity waveforms of the main jet so that pulsed liquid jets were generated, and the simulation for a destruction behavior of the soft elastic body when the pulsed liquid jets were ejected onto the soft elastic body was performed.

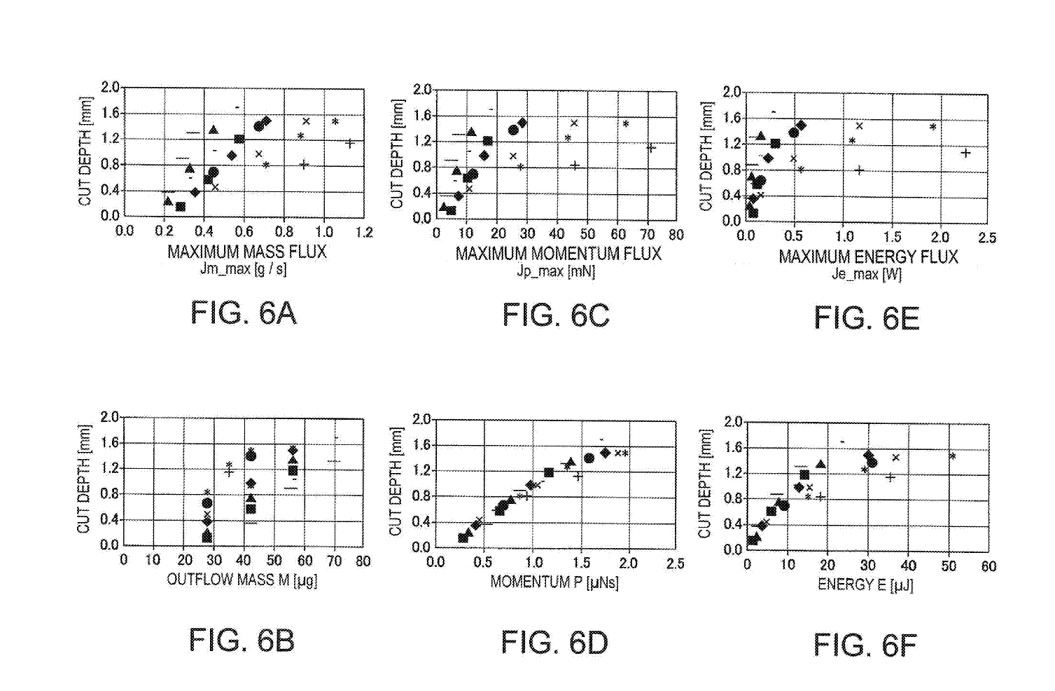

FIG. 6 are diagrams respectively plotting simulation results in a case where a longitudinal axis expresses a cut depth of a cutting target object, and a transverse axis expresses the maximum mass flow flux Jm_max (FIG. 6(a)), the outflow mass M (FIG. 6(b)), the maximum momentum flow flux Jp_max (FIG. 6(c)), the momentum P (FIG. 6(d)), the maximum energy flow flux Je_max (FIG. 6(e)), and the energy E (FIG. 6(f)). In FIG. 6, a simulation result obtained when a sine wave with the duration of 63 μs is provided as a flow velocity waveform of the main jet is indicated by a plot of “*”; a simulation result obtained when a sine wave with the duration of 125 μs is provided as a flow velocity waveform of the main jet is indicated by a plot of “♦”; and a simulation result obtained when a sine wave with the duration of 200 μs is provided as a flow velocity waveform of the main jet is indicated by a plot of “−”. In addition, a simulation result obtained when a triangular wave with the duration of 63 μs is provided as a flow velocity waveform of the main jet is indicated by a plot of “+”; a simulation result obtained when a triangular wave with the duration of 125 μs is provided as a flow velocity waveform of the main jet is indicated by a plot of “X”; and a simulation result obtained when a triangular wave with the duration of 200 μs is provided as a flow velocity waveform of the main jet is indicated by a plot of a square shape displayed black. Further, a simulation result obtained when a rectangular wave with the duration of 63 μs is provided as a flow velocity waveform of the main jet is indicated by a plot of “”; a simulation result obtained when a rectangular wave with the duration of 125 μs is provided as a flow velocity waveform of the main jet is indicated by a plot of a triangular shape displayed black; and a simulation result obtained when a rectangular wave with the duration of 200 μs is provided as a flow velocity waveform of the main jet is indicated by a plot of “−”.

As illustrated in FIGS. 6(a), 6(c) and 6(e), the relationship between each of the three parameters such as the maximum mass flow flux Jm_max, the maximum momentum flow flux Jp_max, and the maximum energy flow flux Je_max, and the cut depth greatly varies depending on the shape of the waveform provided as a flow velocity waveform of the main jet, and thus it was found that a mutual correlation is low. Especially, this suggests that the mass flow flux has a value proportional to a flow velocity, and thus a cut depth is not defined by only the maximum flow velocity of the main jet.

Next, regarding the relationship between each of the three parameters such as the outflow mass M, the momentum P, and the energy E, illustrated in FIGS. 6(b), 6(d) and 6(f), and the cut depth, the relationship between the outflow mass M and the cut depth greatly varies depending on the shape of the waveform provided as a flow velocity waveform of the main jet, and thus a mutual correlation is low. In contrast, in the relationship with the momentum P or the energy E, a variation due to the shape of the provided waveform is small, and the respective plots are substantially distributed on the same curve. Of the momentum P and the energy E, the momentum P less varies. Therefore, it can be said that the cut depth has a high correlation with the momentum P or the energy E, and is highly correlated with, especially, the momentum P.

Here, the simulation was performed in a case where the diameter of the liquid ejection opening was 0.15 mm, and a standoff distance was 0.5 mm, but simulation was performed for other liquid ejection opening diameters or standoff distances, and it was found that a quantitative tendency that the cut depth is highly correlated with the momentum P or the energy E does not greatly change.

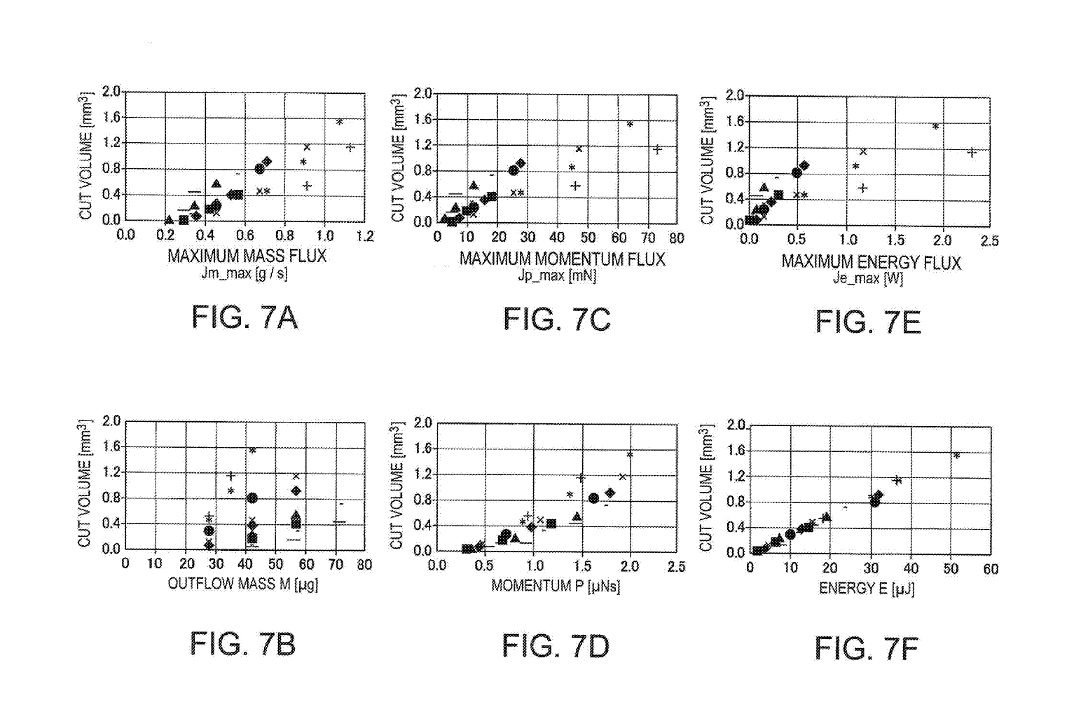

FIG. 7 are diagrams respectively plotting simulation results in a case where a longitudinal axis expresses a cut volume of a cutting target object, and a transverse axis expresses the maximum mass flow flux Jm_max (FIG. 7(a)), the outflow mass M (FIG. 7(b)), the maximum momentum flow flux Jp_max (FIG. 7(c)), the momentum P (FIG. 7(d)), the maximum energy flow flux Je_max (FIG. 7(e)), and the energy E (Fig. (f)). Relationships between waveforms provided as a flow velocity waveform of the main jet and the types of plots are the same as in FIG. 6.

As illustrated in FIGS. 7(a), 7(c) and 7(e), the relationship between each of the three parameters such as the maximum mass flow flux Jm_max, the maximum momentum flow flux Jp_max, and the maximum energy flow flux Je_max, and the cut volume varies depending on the shape of the waveform provided as a flow velocity waveform of the main jet although not as much as the relationship with the cut depth, and thus it is considered that a mutual correlation is low.

Next, regarding the relationship between each of the three parameters such as the outflow mass M, the momentum P, and the energy E, illustrated in FIGS. 7(b), 7(d) and 7(f), and the cut volume, the relationship between the outflow mass M and the cut volume greatly varies depending on the shape of the waveform provided as a flow velocity waveform of the main jet in the same manner as in the cut depth, and thus a mutual correlation is low. In contrast, in the relationship with the momentum P or the energy E, a variation due to the shape of the provided waveform is small in the same manner as in the cut depth, and the respective plots are substantially distributed on the same curve. The energy E less varies than the momentum P. Therefore, it can be said that the cut volume has a high correlation with the momentum P or the energy E, and is highly correlated with, especially, the energy E.

Here, the simulation was performed in a case where the diameter of the liquid ejection opening was 0.15 mm, and a standoff distance was 0.5 mm, but simulation was performed for other liquid ejection opening diameters or other standoff distances, and it was found that a quantitative tendency that the cut volume is highly correlated with the momentum P or the energy E does not greatly change.

In the present embodiment, on the basis of the above examination results, simulation for representative drive voltage waveforms which are actually applied to the piezoelectric element 45 is performed in advance, and thus correspondence relationships among the momentum P, the energy E, rising frequencies, and voltage amplitudes are acquired. During surgery, corresponding rising frequency and voltage amplitude are specified in response to an operation of changing the momentum P or the energy E by an operator, and driving of the piezoelectric element 45 is controlled.

First, the flow velocity waveform of the main jet is obtained through simulation by providing a drive voltage waveform in which the rising frequency is changed in steps in a state in which the voltage amplitude is fixed. Similarly, the flow velocity waveform of the main jet is obtained through simulation by providing a drive voltage waveform in which the voltage amplitude is changed in steps in a state in which the rising frequency is fixed. The simulation may be performed, for example, by using numerical value simulation which is based on a model replacing a channel system of the liquid ejection device with fluid (channel) resistance, fluid inertance, fluid compliance, or the like, and which uses an equivalent circuit method. Alternatively, a finite element method (FEM), a finite volume method (FVM), or the like may be used.

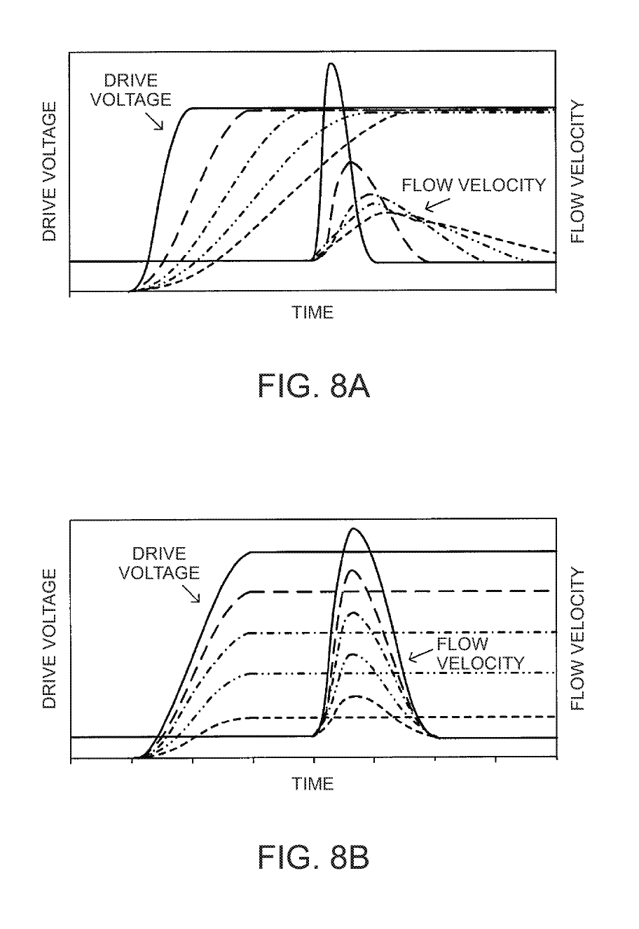

FIG. 8(a) is a diagram illustrating simulation results of the flow velocity waveform of the main jet in a case of changing the rising frequency. As illustrated in FIG. 8(a), if the rising frequency is low (the rising time Tpr is long), in the flow velocity waveform of the main jet, a rising timing does not vary, and the duration is lengthened, and thus the amplitude (the maximum value of the flow velocity) thereof is also reduced. FIG. 8(b) is a diagram illustrating simulation results of the flow velocity waveform of the main jet in a case of changing the voltage amplitude. As illustrated in FIG. 8(b), if the voltage amplitude is reduced, in the flow velocity waveform of the main jet, the duration is maintained unlike in the cases where the rising frequency is reduced, and the waveform amplitude (the maximum value of the flow velocity) is reduced.

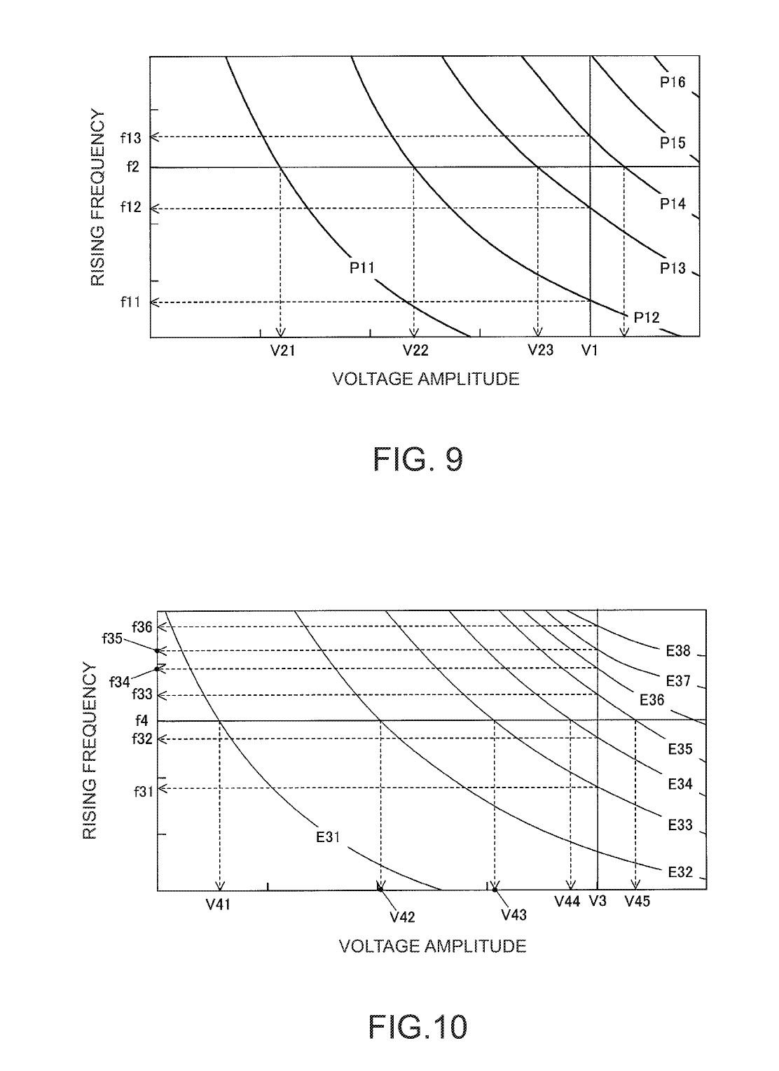

Next, the momentum P and the energy E are obtained for each of the obtained flow velocity waveforms of the main jet. FIG. 9 is a diagram illustrating correspondence relationships among the momentum P, the rising frequency, and the voltage amplitude for each of the obtained flow velocity waveform of the main jet. FIG. 9 is obtained by plotting the obtained momentum P in a coordinate space in which a longitudinal axis expresses the rising frequency, and a transverse axis expresses the voltage amplitude, and drawing contour lines regarding the momentum P. The respective contour lines are low on the lower left side in FIG. 9, and increase by a predetermined amount toward the upper right side.

FIG. 10 is a diagram illustrating correspondence relationships among the energy E, the rising frequency, and the voltage amplitude for each of the obtained flow velocity waveform of the main jet. Also in a case of the energy E, FIG. 10 is obtained by plotting the obtained energy E in a coordinate space in which a longitudinal axis expresses the rising frequency, and a transverse axis expresses the voltage amplitude, and drawing contour lines regarding the energy E. The respective contour lines are low on the lower left side in FIG. 10, and increase by a predetermined amount toward the upper right side.

Here, it is noted that gaps between the contour lines are not the same as each other in either case of the momentum P and the energy E, and the momentum P or the energy E does not linearly change in the coordinate axis direction. For example, in the correspondence relationships among the momentum P, the rising frequency, and the voltage amplitude illustrated in FIG. 9, a case is assumed that the voltage amplitude is fixed (to V5, for example), a drive voltage waveform for the piezoelectric element 45 is controlled by changing the rising frequency. In a case where an amount of the momentum P to be changed is to be constant, a frequency change between the rising frequencies f11 and f12 is necessary between the momentums P12 and P13, and a frequency change between the rising frequencies f12 and f13 is necessary between the momentums P13 and P14. However, a frequency gap between the rising frequencies f11 and f12 is different from a frequency gap between the rising frequencies f12 and f13. This phenomenon notably appears as the momentum P increases. Therefore, in a case of performing an operation of changing the rising frequency by a predetermined amount in a state in which the voltage amplitude is fixed, the momentum P is not changed as expected, and thus it can be said that a situation may occur in which a cut depth or a cut volume is not changed as intended or perceived by an operator. This may also be the same for a case of performing an operation of changing the voltage amplitude by a predetermined amount in a state in which the rising frequency is fixed. This is also the same for the energy E.

Therefore, in the present embodiment, in the same specification as that of the related art, an operation performed by the operator during surgery is an operation of changing the rising frequency or the voltage amplitude using the lever switch 813. In other words, the operator performs an operation of changing the rising frequency in a state in which the voltage amplitude is fixed, or an operation of changing the voltage amplitude in a state in which the rising frequency is fixed. Furthermore, when the lever switch 813 is moved by one scale, control is performed so that an amount of the momentum P or the energy E to be changed is constant. Specifically, an indication value of the rising frequency (rising frequency indication value) or an indication value of the voltage amplitude (voltage amplitude indication value) is allocated to each lever position. Correspondence relationships among the momentum P or the energy E, the rising frequency, and the voltage amplitude are generated as a data table.

Details thereof will be described.

First, (1) a case is assumed in which an operation of changing the rising frequency using the lever switch 813 is performed with respect to the momentum P. For example, in a case where the voltage amplitude is fixed to V1 illustrated in FIG. 9, rising frequencies corresponding to intersections between the voltage amplitude V1 and the respective contour lines are allocated to lever positions of the lever switch 813 as frequency indication values f11, f12, f13, . . . A data table is created in which the corresponding momentum P is correlated with each combination of the rising frequency indication values and the fixed voltage amplitude. For example, the momentum P12 is correlated with a combination between the rising frequency indication value f11 and the voltage amplitude V1, and the momentum P13 is correlated with a combination between the rising frequency indication value f12 and the voltage amplitude V1. In the above-described way, an amount of the momentum P to be changed when the lever position is moved by one scale can be made constant.

Next, (2) a case is assumed in which an operation of changing the voltage amplitude using the lever switch 813 is performed with respect to the momentum P. For example, in a case where the rising frequency is fixed to f2 illustrated in FIG. 9, voltage amplitudes corresponding to intersections between the rising frequency f2 and the respective contour lines are allocated to lever positions of the lever switch 813 as amplitude indication values V21, V22, V23, . . . A data table is created in which the corresponding momentum P is correlated with each combination of the voltage amplitude indication values and the fixed rising frequency. For example, the momentum P11 is correlated with a combination between the voltage amplitude indication value V21 and the rising frequency f2, and the momentum P12 is correlated with a combination between the voltage amplitude indication value V22 and the rising frequency f2. Also in this case, an amount of the momentum P to be changed when the lever position is moved by one scale can be made constant.

Next, (3) a case is assumed in which an operation of changing the rising frequency using the lever switch 813 is performed with respect to the energy E. For example, in a case where the voltage amplitude is fixed to V3 illustrated in FIG. 10, rising frequencies corresponding to intersections between the voltage amplitude V3 and the respective contour lines are allocated to lever positions of the lever switch 813 as frequency indication values f31, f32, f33, . . . A data table is created in which the corresponding energy E is correlated with each combination of the rising frequency indication values and the fixed voltage amplitude. For example, the energy E33 is correlated with a combination between the rising frequency indication value f31 and the voltage amplitude V3, and the energy E34 is correlated with a combination between the rising frequency indication value f32 and the voltage amplitude V3. In the above-described way, an amount of the energy E to be changed when the lever position is moved by one scale can be made constant.

Next, (4) a case is assumed in which an operation of changing the voltage amplitude using the lever switch 813 is performed with respect to the energy E. For example, in a case where the rising frequency is fixed to f4 illustrated in FIG. 10, voltage amplitudes corresponding to intersections between the rising frequency f4 and the respective contour lines are allocated to lever positions of the lever switch 813 as amplitude indication values V41, V42, V43, . . . A data table is created in which the corresponding energy E is correlated with each combination of the voltage amplitude indication values and the fixed rising frequency. For example, the energy E31 is correlated with a combination between the voltage amplitude indication value V41 and the rising frequency f4, and the energy E32 is correlated with a combination between the voltage amplitude indication value V42 and the rising frequency f4. Also in this case, an amount of the energy E to be changed when the lever position is moved by one scale can be made constant.

Hereinafter, as examples of the present embodiment, the case of (1), the case of (2), the case of (3), and the case of (4) will be respectively described as Example 1, Example 2, Example 3, and Example 4 in this order.

### Example 1

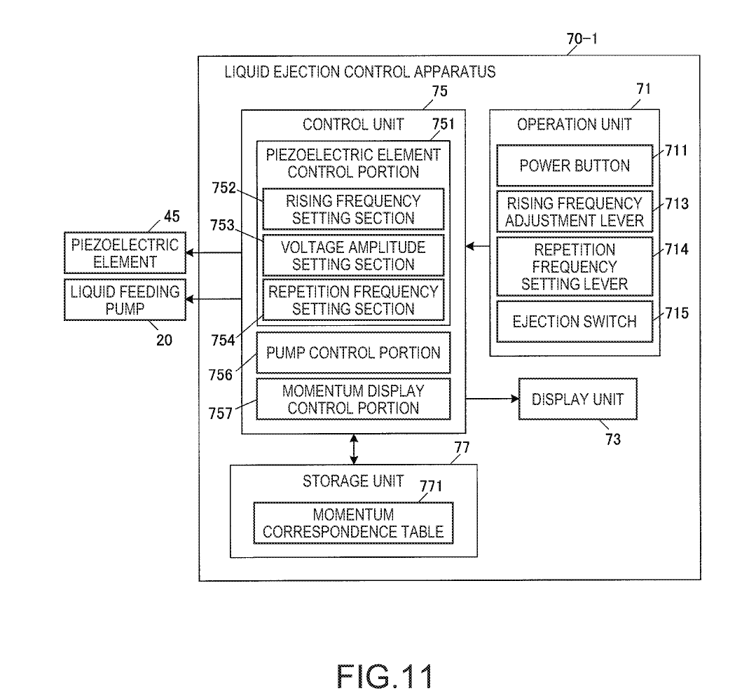

First, Example 1 will be described. FIG. 11 is a block diagram illustrating a functional configuration example of a liquid ejection control apparatus in Example 1. As illustrated in FIG. 11, a liquid ejection control apparatus 70-1 includes an operation unit 71, a display unit 73, a control unit 75, and a storage unit 77.

The operation unit 71 is implemented by various switches such as a button switch, a lever switch, a dial switch, and a pedal switch, and an input device such as a touch panel, a track pad, and a mouse, and outputs an operation signal corresponding to an input operation to the control unit 75. The operation unit 71 includes a power button 711 implemented by the button switch 811 illustrated in FIG. 1, a rising frequency adjustment lever 713 implemented by the lever switch 813 illustrated in FIG. 1, a repetition frequency setting lever 714 implemented by the lever switch 814 illustrated in FIG. 1, and an ejection switch 715 implemented by the pedal switch 83 illustrated in FIG. 1.

The rising frequency adjustment lever 713 is used to input a rising frequency indication value. The operator operates the rising frequency adjustment lever 713, that is, the lever switch 813 illustrated in FIG. 1, so as to select the lever positions with scales such as “1” to “5”, and thus performs an operation of changing a rising frequency in five steps. In Example 1, the rising frequency indication values at the respective lever positions are allocated so that the momentum P is increased by a predetermined level in proportion to a numerical value of a corresponding scale. The number of steps of the lever positions is not limited to five steps, and may be set as appropriate, for example, three steps such as “large”, “intermediate”, and “small”.

The repetition frequency setting lever 714 is used to set a repetition frequency. The operator operates the lever switch 814 illustrated in FIG. 1 so as to select lever positions with scales such as “1” to “5”, and thus performs an operation of changing a repetition frequency (for example, several tens to several hundreds of Hz) of a drive voltage which is repeatedly applied to the piezoelectric element 45, in five steps. Repetition frequencies corresponding to numerical values of the scales are allocated to the respective lever positions. The number of steps of the lever positions is not limited to five steps, and may be set as appropriate.

The display unit 73 is implemented by a display device such as a liquid crystal display (LCD) or an electroluminescence (EL) display, and displays various screens such as a setting screen on the basis of display signals input from the control unit 75. The display unit 73 corresponds to, for example, the liquid crystal monitor 82 illustrated in FIG. 1.

The control unit 75 is implemented by a microprocessor such as a central processing unit (CPU) or a digital signal processor (DSP), and a control device and a calculation device such as an application specific integrated circuit (ASIC), and generally controls the respective portions of the liquid ejection system 1. The control unit 75 includes a piezoelectric element control portion 751, a pump control portion 756, and a momentum display control portion 757. The respective portions constituting the control unit 75 may be formed of hardware such as a dedicated module circuit.

The piezoelectric element control portion 751 includes a rising frequency setting section 752 which sets a rising frequency according to a lever position of the rising frequency adjustment lever 713, a voltage amplitude setting section 753 which sets a voltage amplitude, and a repetition frequency setting section 754 which sets a repetition frequency according to a lever position of the repetition frequency setting lever 714. The piezoelectric element control portion 751 generates a drive voltage waveform, applies a drive signal having the generated waveform to the piezoelectric element 45, and, at this time, generates the drive voltage waveform according to a rising frequency set by the rising frequency setting section 752 and a voltage amplitude set by the voltage amplitude setting section 753. In Example 1, the voltage amplitude set by the voltage amplitude setting section 753 is fixed.

The pump control portion 756 outputs a drive signal to the liquid feeding pump 20 so as to derive the liquid feeding pump 20. The momentum display control portion 757 performs control of displaying, on the display unit 73, a frequency indication value (that is, the present value of a rising frequency) allocated to a currently selected lever position of the rising frequency adjustment lever 713, and the corresponding momentum P (that is, the present value of the momentum P).

The storage unit 77 is implemented by various integrated circuit (IC) memories such as a read only memory (ROM), a flash ROM, or a random access memory (RAM), or a storage medium such as a hard disk. The storage unit 77 stores in advance a program for operating the liquid ejection system 1 and thus realizing various functions of the liquid ejection system 1, data used during execution of the program, and the like, or temporarily stores data whenever a process is performed.

The storage unit 77 stores a momentum correspondence table 771. The momentum correspondence table 771 is a data table, described with reference to FIG. 9, in which correspondence relationships among the momentum P, the rising frequency (rising frequency indication values), and the voltage amplitude (which is fixed in Example 1) are set.

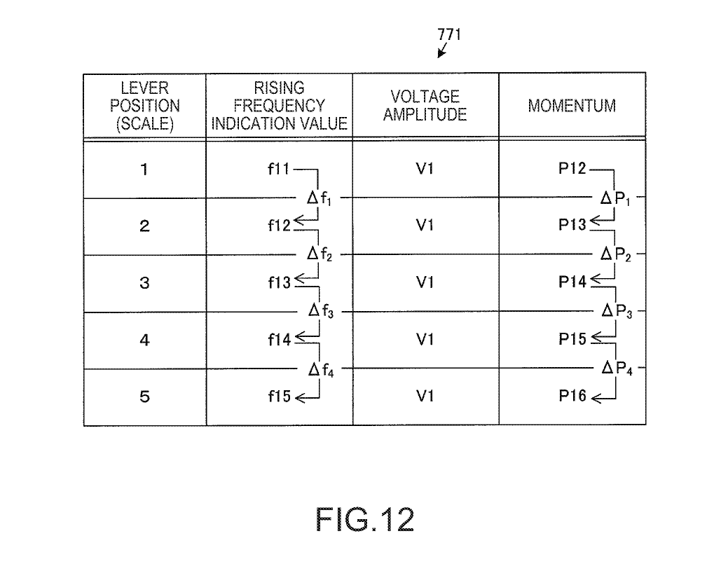

FIG. 12 is a diagram illustrating a data configuration example of the momentum correspondence table 771. As illustrated in FIG. 12, in the momentum correspondence table 771, the momentum P, a frequency indication value allocated to a corresponding lever position, and voltage amplitude are set in correlation with the lever position (scale). Voltage amplitudes are all set to the same value V1. On the other hand, the rising frequency indication values are defined so that change amounts ΔP₁, ΔP₂, . . . of the momentum P among the adjacent lever positions are constant, and thus change amounts Δf₁, Δf₂, . . . of the rising frequencies are not necessarily constant. In a case where the rising frequency adjustment lever 713 is operated, the rising frequency setting section 752 reads a rising frequency indication value at a selected lever position from the momentum correspondence table 771, and sets a rising frequency.

### [Flow of Process]

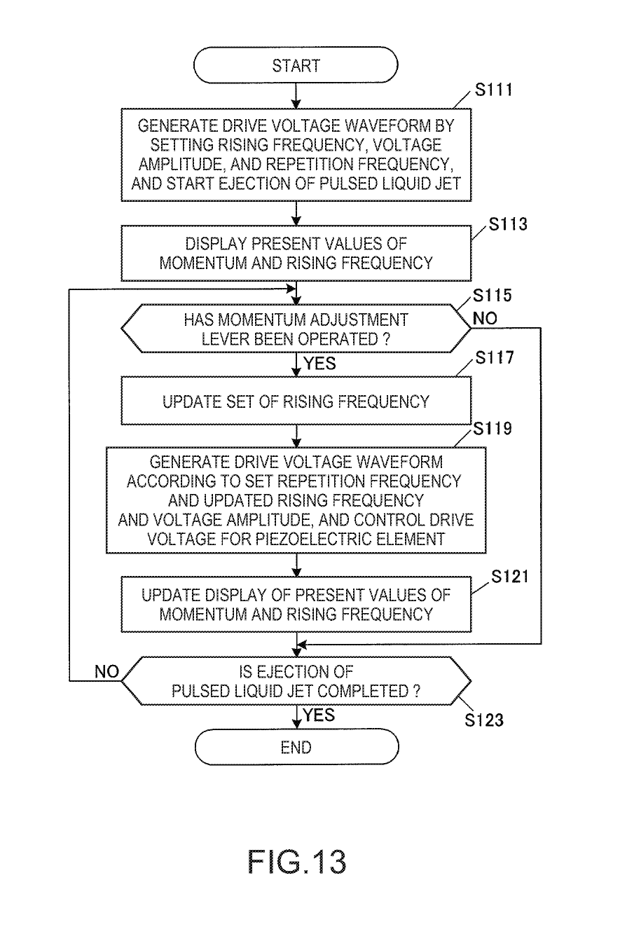

FIG. 13 is a flowchart illustrating a flow of a process performed by the control unit 75 when a pulsed liquid jet is ejected. As illustrated in FIG. 13, if the power button 711 is operated so that power is supplied to the liquid ejection control apparatus 70-1, and thus an instruction for starting of ejection of a pulsed liquid jet is given by the ejection switch 715, the pump control portion 756 drives the liquid feeding pump 20, and the piezoelectric element control portion 751 drives the piezoelectric element 45 so as to start ejection of the pulsed liquid jet (step S111). At this time, the rising frequency setting section 752 acquires a currently selected lever position of the rising frequency adjustment lever 713, reads a rising frequency indication value from the momentum correspondence table 771, and sets a rising frequency. The voltage amplitude setting section 753 reads voltage amplitude which is set to a fixed value from the momentum correspondence table 771, and sets voltage amplitude. The repetition frequency setting section 754 acquires a currently selected lever position of the repetition frequency setting lever 714, and sets a repetition frequency. The piezoelectric element control portion 751 generates a drive voltage waveform according to the set rising frequency and voltage amplitude, and applies a drive signal having the generated drive voltage waveform to the piezoelectric element 45.

The momentum display control portion 757 performs control of reading the momentum P at the acquired lever position from the momentum correspondence table 771, and displaying the read momentum P on the display unit 73 along with the rising frequency set in step S111 (step S113).

Thereafter, the control unit 75 monitors an operation on the rising frequency adjustment lever 713 in step S115 until it is determined that ejection of a pulsed liquid jet is finished through an operation on the ejection switch 715 (NO in step S123). In a case where the rising frequency adjustment lever 713 is operated (YES in step S115), the rising frequency setting section 752 reads a frequency indication value at a selected lever position from the momentum correspondence table 771, and updates the set rising frequency (step S117). Next, the piezoelectric element control portion 751 generates a drive voltage waveform according to the rising frequency set in step S117 and the voltage amplitude set in step S111, and applies a drive signal having the generated drive voltage waveform to the piezoelectric element 45 (step S119).

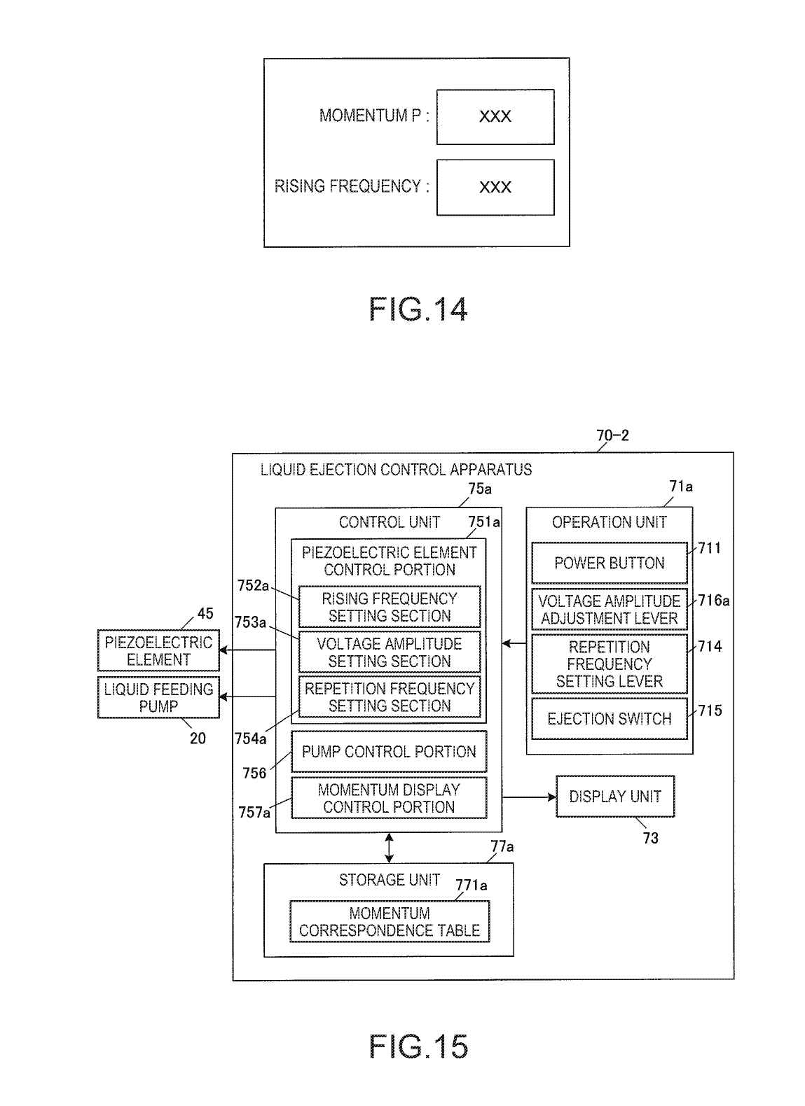

The momentum display control portion 757 reads the momentum P at the selected lever position from the momentum correspondence table 771, and performs control of updating the display of the display unit 73 according to the read momentum P and the rising frequency set in step S117 (step S121). FIG. 14 is a diagram illustrating a display screen example which is displayed in step S113 and is updated and displayed in step S121. The operator can perform work on the basis of the display screen during surgery while recognizing the present value of the momentum P related to a pulsed liquid jet ejected from the liquid ejection opening 61, and the present value of the rising frequency used for control thereof. Display of a momentum indication value is not limited to the display in numerical values illustrated in FIG. 14, and a value may be displayed in a meter form, or a change in the momentum P due to a changing operation from starting of ejection of a pulsed liquid jet may be displayed in a graph form.

The momentum display control portion 757 may display not only the momentum P and a rising frequency indication value but also a repetition frequency on the display unit 73. The present voltage amplitude may also be displayed.

According to Example 1, it is possible to control a drive voltage waveform for the piezoelectric element 45 according to a rising frequency which is optimal for achieving a cut depth and a cut volume corresponding to an operation sense on the basis of preset correspondence relationships among the momentum P, a rising frequency, and predetermined voltage amplitude. For example, since the momentum P is changed by an amount corresponding to a scale interval if the rising frequency adjustment lever 713 is moved by one scale, it is possible to set a cut depth or a cut volume to be suitable for a user's sense or intention and thus to improve convenience.

### Example 2

Next, Example 2 will be described. The same constituent elements as in Example 1 are given the same reference numerals. FIG. 15 is a block diagram illustrating a functional configuration example of a liquid ejection control apparatus in Example 2. As illustrated in FIG. 15, a liquid ejection control apparatus 70-2 includes an operation unit 71a, a display unit 73, a control unit 75a, and a storage unit 77a.

The operation unit 71a includes a voltage amplitude adjustment lever 716a implemented by the lever switch 813 illustrated in FIG. 1. The voltage amplitude adjustment lever 716a is used to input a voltage amplitude indication value. The operator operates the voltage amplitude adjustment lever 716a, that is, the lever switch 813 illustrated in FIG. 1, so as to select the lever positions with scales such as “1” to “5”, and thus performs an operation of changing voltage amplitude in five steps. In Example 2, the voltage amplitude indication values at the respective lever positions are allocated so that the momentum P is increased by a predetermined level in proportion to a numerical value of a corresponding scale.

The control unit 75a includes a piezoelectric element control portion 751a, a pump control portion 756, and a momentum display control portion 757a.

The piezoelectric element control portion 751a includes a rising frequency setting section 752a which sets a rising frequency, a voltage amplitude setting section 753a which sets a voltage amplitude according to a lever position of the voltage amplitude adjustment lever 716a, and a repetition frequency setting section 754a which sets a repetition frequency according to a lever position of the repetition frequency setting lever 714. The piezoelectric element control portion 751a generates a drive voltage waveform, applies a drive signal having the generated waveform to the piezoelectric element 45, and, at this time, generates the drive voltage waveform according to a rising frequency set by the rising frequency setting section 752a and a voltage amplitude set by the voltage amplitude setting section 753a. In Example 2, the rising frequency set by the rising frequency setting section 752a is fixed.

The momentum display control portion 757a performs control of displaying, on the display unit 73, a voltage amplitude indication value (that is, the present value of voltage amplitude) allocated to a currently selected lever position of the voltage amplitude adjustment lever 716a, and the corresponding momentum P (that is, the present value of the momentum P).

The momentum display control portion 757a may display not only the momentum P and a voltage amplitude indication value but also a repetition frequency on the display unit 73. The present rising frequency may also be displayed.

The storage unit 77a stores a momentum correspondence table 771a. The momentum correspondence table 771a is a data table, described with reference to FIG. 9, in which correspondence relationships among the momentum P, the rising frequency (which is fixed in Example 2), and the voltage amplitude (voltage amplitude indication values) are set.

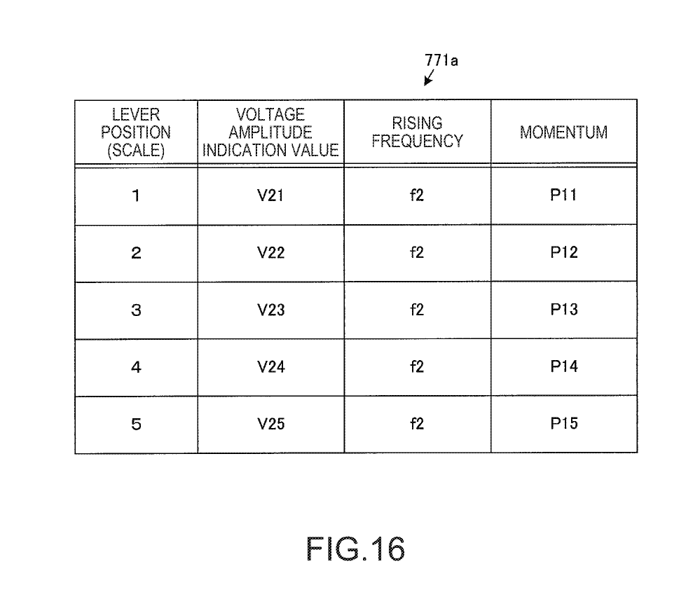

FIG. 16 is a diagram illustrating a data configuration example of the momentum correspondence table 771a. As illustrated in FIG. 16, in the momentum correspondence table 771a, the momentum P, a voltage amplitude indication value allocated to a corresponding lever position, and a rising frequency are set in correlation with the lever position (scale). Rising frequencies are all set to the same value f2. In a case where the voltage amplitude adjustment lever 716a is operated, the voltage amplitude setting section 753a reads a voltage amplitude indication value at a selected lever position from the momentum correspondence table 771a, and sets voltage amplitude. In the same manner as in the momentum correspondence table 771 illustrated in FIG. 12, a change amount (change width) of the momentum P between adjacent lever positions are constant, but a change amount (change width) of amplitude indication values is not necessarily constant.

According to Example 2, it is possible to control a drive voltage waveform for the piezoelectric element 45 according to voltage amplitude which is optimal for achieving a cut depth and a cut volume corresponding to an operation sense on the basis of preset correspondence relationships among the momentum P, voltage amplitude, and a predetermined rising frequency. For example, since the momentum P is changed by an amount corresponding to a scale interval if the voltage amplitude adjustment lever 716a is moved by one scale, it is possible to set a cut depth or a cut volume to be suitable for a user's sense or intention and thus to improve convenience.

### Example 3

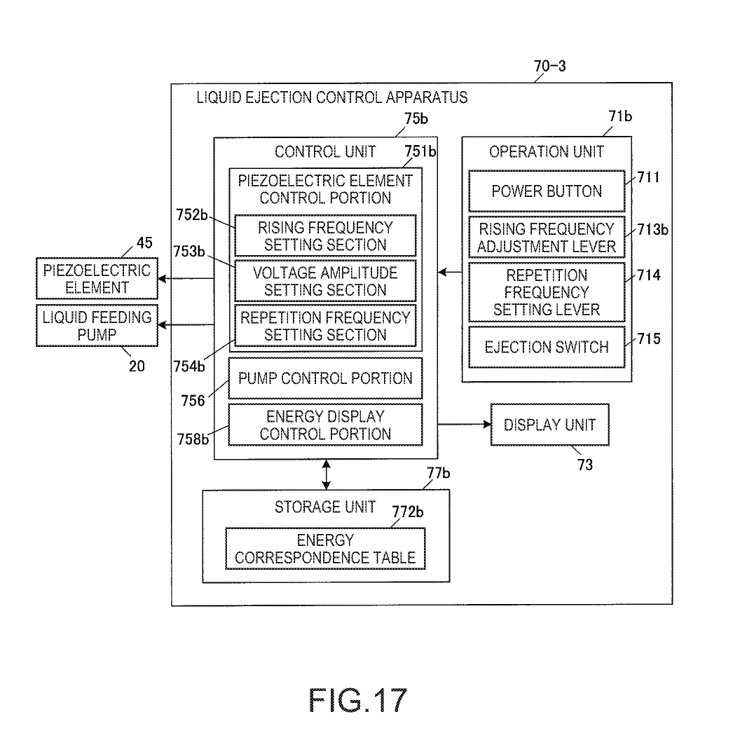

Next, Example 3 will be described. The same constituent elements as in Example 1 are given the same reference numerals. FIG. 17 is a block diagram illustrating a functional configuration example of a liquid ejection control apparatus in Example 3. As illustrated in FIG. 17, a liquid ejection control apparatus 70-3 includes an operation unit 71b, a display unit 73, a control unit 75b, and a storage unit 77b.

The operation unit 71b includes a rising frequency adjustment lever 713b implemented by the lever switch 813 illustrated in FIG. 1. The rising frequency adjustment lever 713b is used to input a rising frequency indication value. The operator operates the rising frequency adjustment lever 713b, that is, the lever switch 813 illustrated in FIG. 1, so as to select the lever positions with scales such as “1” to “5”, and thus performs an operation of changing a rising frequency in five steps. In Example 3, the rising frequency indication values at the respective lever positions are allocated so that the energy E is increased by a predetermined level in proportion to a numerical value of a corresponding scale.

The control unit 75b includes a piezoelectric element control portion 751b, a pump control portion 756, and an energy display control portion 758b.

The piezoelectric element control portion 751b includes a rising frequency setting section 752b which sets a rising frequency according to a lever position of the rising frequency adjustment lever 713b, a voltage amplitude setting section 753b which sets a voltage amplitude, and a repetition frequency setting section 754b which sets a repetition frequency according to a lever position of the repetition frequency setting lever 714. The piezoelectric element control portion 751b generates a drive voltage waveform, applies a drive signal having the generated waveform to the piezoelectric element 45, and, at this time, generates the drive voltage waveform according to a rising frequency set by the rising frequency setting section 752b and a voltage amplitude set by the voltage amplitude setting section 753b. In Example 3, the voltage amplitude set by the voltage amplitude setting section 753b is fixed.

The energy display control portion 758b performs control of displaying, on the display unit 73, a rising frequency indication value (that is, the present value of a rising frequency) allocated to a currently selected lever position of the rising frequency adjustment lever 713b, and the corresponding energy E (that is, the present value of the energy E).

The energy display control portion 758b may display not only the energy E and a rising frequency indication value but also a repetition frequency on the display unit 73. The present voltage amplitude may also be displayed.

The storage unit 77b stores an energy correspondence table 772b. The energy correspondence table 772b is a data table, described with reference to FIG. 10, in which correspondence relationships among the energy E, the rising frequency (frequency indication values), and the voltage amplitude (which is fixed in Example 3) are set.

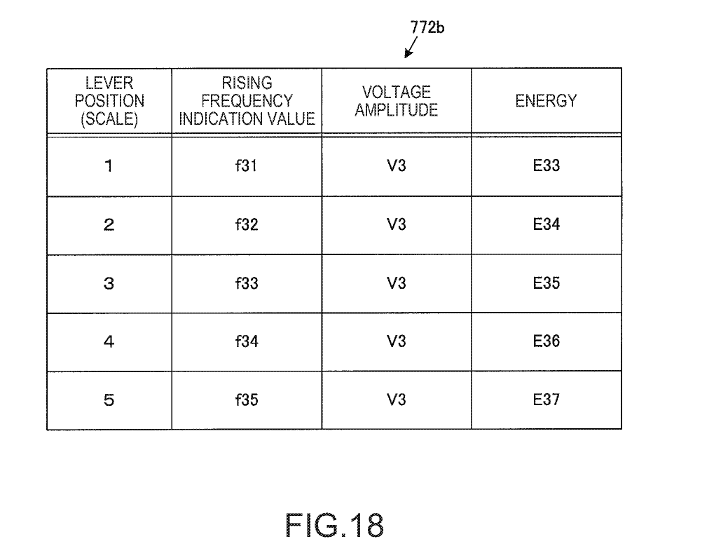

FIG. 18 is a diagram illustrating a data configuration example of the energy correspondence table 772b. As illustrated in FIG. 18, in the energy correspondence table 772b, the energy E, a rising frequency indication value allocated to a corresponding lever position, and voltage amplitude are set in correlation with the lever position (scale). Voltage amplitudes are all set to the same value V3. In a case where the rising frequency adjustment lever 713b is operated, the rising frequency setting section 752b reads a rising frequency indication value at a selected lever position from the energy correspondence table 772b, and sets a rising frequency. In the same manner as in the momentum correspondence table 771 illustrated in FIG. 12, a change amount (change width) of the momentum P between adjacent lever positions are constant, but a change amount (change width) of rising frequency indication values is not necessarily constant.

According to Example 3, it is possible to control a drive voltage waveform for the piezoelectric element 45 according to a rising frequency which is optimal for achieving a cut depth and a cut volume corresponding to an operation sense on the basis of preset correspondence relationships among the energy E, a rising frequency, and predetermined voltage amplitude. For example, since the energy E is changed by an amount corresponding to a scale interval if the rising frequency adjustment lever 713b is moved by one scale, it is possible to set a cut depth or a cut volume to be suitable for a user's sense or intention and thus to improve convenience.

### Example 4

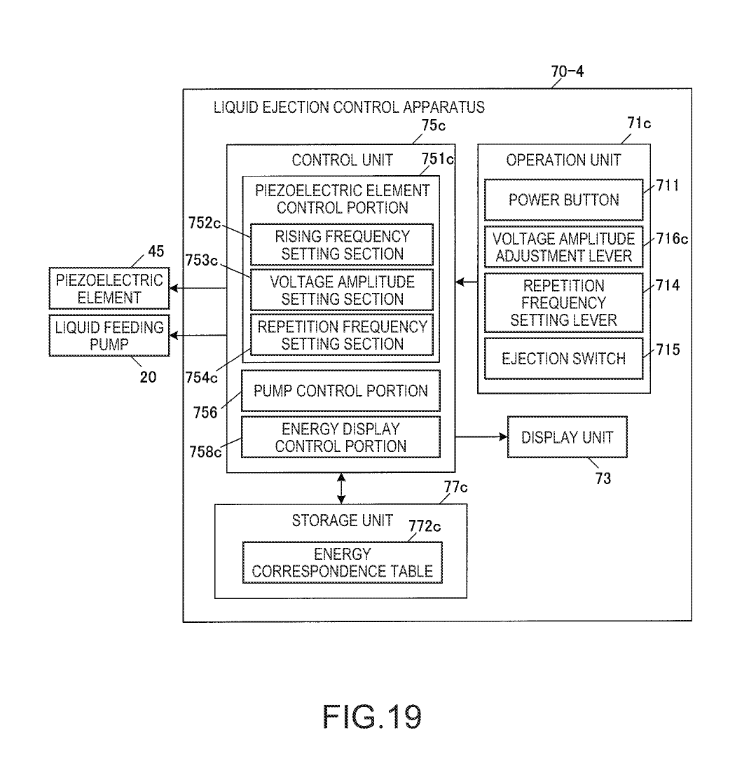

Next, Example 4 will be described. The same constituent elements as in Example 1 are given the same reference numerals. FIG. 19 is a block diagram illustrating a functional configuration example of a liquid ejection control apparatus in Example 4. As illustrated in FIG. 19, a liquid ejection control apparatus 70-4 includes an operation unit 71c, a display unit 73, a control unit 75c, and a storage unit 77c.

The operation unit 71c includes a voltage amplitude adjustment lever 716c implemented by the lever switch 813 illustrated in FIG. 1. The voltage amplitude adjustment lever 716c is used to input a voltage amplitude indication value. The operator operates the voltage amplitude adjustment lever 716c, that is, the lever switch 813 illustrated in FIG. 1, so as to select the lever positions with scales such as “1” to “5”, and thus performs an operation of changing voltage amplitude in five steps. In Example 4, the voltage amplitude indication values at the respective lever positions are allocated so that the energy E is increased by a predetermined level in proportion to a numerical value of a corresponding scale.

The control unit 75c includes a piezoelectric element control portion 751c, a pump control portion 756, and an energy display control portion 758c as a display control portion.

The piezoelectric element control portion 751c includes a rising frequency setting section 752c which sets a rising frequency, and a voltage amplitude setting section 753c which sets a voltage amplitude according to a lever position of the voltage amplitude adjustment lever 716c. The piezoelectric element control portion 751c generates a drive voltage waveform, applies a drive signal having the generated waveform to the piezoelectric element 45, and, at this time, generates the drive voltage waveform according to a rising frequency set by the rising frequency setting section 752c and a voltage amplitude set by the voltage amplitude setting section 753c. In Example 4, the rising frequency set by the rising frequency setting section 752c is fixed.

The energy display control portion 758c performs control of displaying, on the display unit 73, a voltage amplitude indication value (that is, the present value of voltage amplitude) allocated to a currently selected lever position of the voltage amplitude adjustment lever 716c, and the corresponding energy E (that is, the present value of the energy E).

The energy display control portion 758c may display not only the energy E and a voltage amplitude indication value but also a repetition frequency on the display unit 73. The present rising frequency may also be displayed.

The storage unit 77c stores an energy correspondence table 772c. The energy correspondence table 772c is a data table, described with reference to FIG. 10, in which correspondence relationships among the energy E, the rising frequency (which is fixed in Example 4), and the voltage amplitude (voltage amplitude indication values) are set.

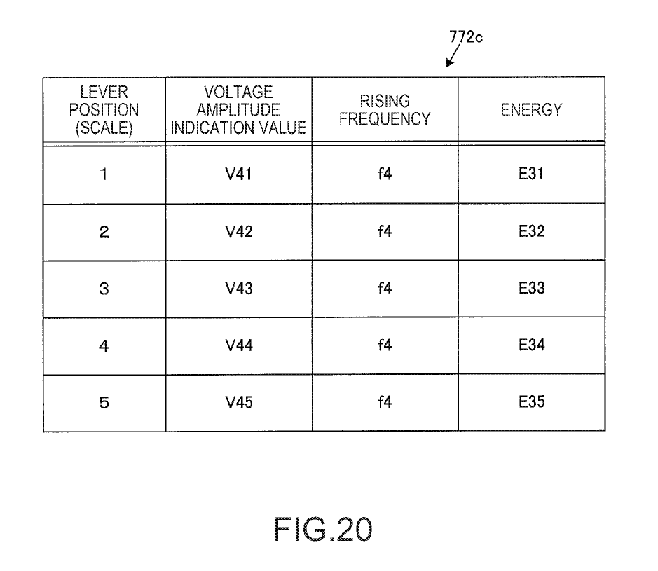

FIG. 20 is a diagram illustrating a data configuration example of the energy correspondence table 772c. As illustrated in FIG. 20, in the energy correspondence table 772c, the energy E, a voltage amplitude indication value allocated to a corresponding lever position, and a rising frequency are set in correlation with the lever position (scale). Rising frequencies are all set to the same value f4. In a case where the voltage amplitude adjustment lever 716c is operated, the voltage amplitude setting section 753c reads a voltage amplitude indication value at a selected lever position from the energy correspondence table 772c, and sets voltage amplitude. In the same manner as in the momentum correspondence table 771 illustrated in FIG. 12, a change amount (change width) of the energy between adjacent lever positions are constant, but a change amount (change width) of amplitude indication values is not necessarily constant.

According to Example 4, it is possible to control a drive voltage for the piezoelectric element 45 according to voltage amplitude which is optimal for achieving a cut depth and a cut volume corresponding to an operation sense on the basis of preset correspondence relationships among the energy E, voltage amplitude, and a predetermined rising frequency. For example, since the energy E is changed by an amount corresponding to a scale interval if the voltage amplitude adjustment lever 716c is moved by one scale, it is possible to set a cut depth or a cut volume to be suitable for a user's sense or intention and thus to improve convenience.

As mentioned above, the embodiment including four Examples has been described, but an embodiment of the invention is not limited to the above-described embodiment.

For example, the momentum correspondence tables 771 and 771a and the energy correspondence table 772b and 772c may be prepared (stored in the storage unit 77, 77a, 77b and 77c) for respective types of liquid ejection device 30, and the momentum correspondence tables 771 or the energy correspondence table 772 corresponding to the type of liquid ejection device 30 may be selectively used. For example, it is preferable to prepare tables for respective types of liquid ejection device 30 in which structures related to ejection of a pulsed liquid jet are different from each other, such as differences in inner diameters and lengths of the liquid ejection opening 61 and the nozzle 60, differences in an inner diameter and a length of the ejection tube 50, differences in characteristics of the piezoelectric element 45, and a difference in a volume of the pressure chamber 44. This is because the liquid ejection device 30 may be replaced with a different type thereof depending on a cutting target object, for example, an affected part in a case where the liquid ejection system is used for a surgery application, depending on the type of food in a case where the liquid ejection system is used for a food processing application, depending on the kind of each material in a case where the liquid ejection system is used for a cutting processing application of a gel material, or a resin material such as rubber or plastic, that is, depending on a shape, a destruction threshold value, and elasticity and viscosity of a target material to be cut and processed.

More preferably, information indicating the type of liquid ejection device 30 is stored in the liquid ejection device, and the liquid ejection control apparatus 70 reads the information from the liquid ejection device 30 connected thereto, and automatically switch between the momentum correspondence tables 771 and 771a and the energy correspondence tables 772b and 772c.

In the above-described embodiment, a rising frequency has been exemplified as an index value related to rising. In contrast, the rising time Tpr may be used instead of the rising frequency.

The rising frequency adjustment levers 713 and 713b or the voltage amplitude adjustment levers 716a and 716c are not limited to case of being implemented by the lever switch 813, and may be implemented by, for example, a dial switch or a button switch. The levers may be implemented by a key switch based on software in a case where the display unit 73 is a touch panel. In this case, a user performs a touch operation on the touch panel as the display unit 73 so as to perform an operation of inputting an indication value of a rising frequency or voltage amplitude.

1. A liquid ejection control apparatus controlling a liquid ejection device which ejects a liquid in a pulse form by using a piezoelectric element, the liquid ejection control apparatus comprising:
an operation unit that is used to perform a changing operation on one of the amplitude of a drive voltage waveform applied to the piezoelectric element, and an index value related to rising of the drive voltage waveform; and
a control unit that performs control of setting a value of the one in response to an input operation on the operation unit so as to make constant a change amount regarding momentum or kinetic energy related to a pulsed liquid jet ejected from the liquid ejection device and the change amount per unit operation level for the operation unit in a state in which the other of the amplitude of the drive voltage waveform and the index value related to rising of the drive voltage waveform is set to a predetermined value.

2. The liquid ejection control apparatus according to claim 1, further comprising:
a display control unit that performs control of displaying the present value of momentum and kinetic energy related to the pulsed liquid jet.

3. The liquid ejection control apparatus according to claim 1,
wherein a correspondence relationship between the amplitude of the drive voltage waveform applied to the piezoelectric element and the index value related to rising of the drive voltage waveform, causing the change amount per unit operation level to be constant, is defined for each type of liquid ejection device, and
wherein the control unit performs control on the basis of the correspondence relationship corresponding to the type of liquid ejection device.

4. The liquid ejection control apparatus according to claim 1,
wherein the liquid ejection device is controlled so that momentum of the pulsed liquid jet is equal to or less than 0.1 millinewton seconds (mNs), or kinetic energy of the pulsed liquid jet is equal to or less than 100 millijoules (mJ).

5. The liquid ejection control apparatus according to claim 1,
wherein the liquid ejection device is controlled so that a living tissue is cut with the pulsed liquid jet.

6. The liquid ejection control apparatus according to claim 1,
wherein the index value related to the rising is represented by time or a frequency related to the rising of the drive voltage waveform.

7. (canceled)

8. A control method for a liquid ejection device which ejects a liquid in a pulse form by using a piezoelectric element, the control method comprising:
detecting an input operation on an operation unit that is used to perform a changing operation on one of the amplitude of a drive voltage waveform applied to the piezoelectric element, and an index value related to rising of the drive voltage waveform; and
setting a value of the one in response to an input operation on the operation unit so as to make constant a change amount regarding momentum or kinetic energy related to a pulsed liquid jet ejected from the liquid ejection device and the change amount per unit operation level for the operation unit in a state in which the other of the amplitude of the drive voltage waveform and the index value related to rising of the drive voltage waveform is set to a predetermined value.
